(19) United States
(12) Patent Application Publication
(10) Pub. No.: US 2008/0188792 A1
(43) Pub. Date: Aug. 7, 2008

(54) Phacoemulsification Needle

(76) Inventor: Graham David Barrett, Western Australia (AU)

Correspondence Address: PEARNE & GORDON LLP, 1801 EAST 9TH STREET, SUITE 1200, CLEVELAND, OH, 44114-3108, US

(21) Appl. No.: 10/599,166

(22) PCT Filed: Mar. 24, 2005
(86) PCT No.: PCT/AU05/00422

§ 371 Date: Sep. 21, 2006

(30) Foreign Application Priority Data
Mar. 25, 2004 (AU) 2004901550
Sep. 17, 2004 (AU) 2004905374

(51) Int. Cl. A61N 7/00; A61M 1/00

(52) U.S. Cl. 604/22; 604/35

(57) ABSTRACT

A phacoemulsification needle is provided having a rod member (12) arranged for transmission of ultrasonic energy to an ocular substrate to effect emulsification thereof, and a hollow tube member (14) having an inner surface (18) defining a lumen arranged for aspiration of emulsified ocular material, the hollow tube member (14) being disposed about the rod member (12), the rod member (12) having a distal end (11) and a proximal end (13) and the hollow tube member (14) having a distal end (19) and a proximal end (17).

### FIELD OF THE INVENTION

The present invention relates to a phacoemulsification needle.

### BACKGROUND OF THE INVENTION

The crystalline lens of the human eye transmits and focuses light and is located behind the iris attached to the wall of the eye by suspensory ligaments known as the zonules. The lens consists of a more rigid central nucleus surrounded by peripheral cortical material, which has a softer consistency. A fine membrane known as the capsule contains the entire lens.

Cataract formation refers to a loss of transparency of the crystalline lens of the eye and is a common occurrence with age. This results in a progressive reduction in vision, which can be restored with surgery.

Modem cataract surgery involves removal of the cataractous lens and insertion of a plastic intraocular lens to replace the crystalline lens. Modem cataract surgery uses ultrasonic energy to fragment and aspirate the cataractous lens by a technique known as phacoemulsification.

During a phacoemulsification procedure a central opening is made in the anterior portion of the capsule to allow access to the lenticular material by the phacoemulsification needle, which typically has an outer wall and central lumen. A plastic sleeve surrounding the needle provides a conduit for transmission of fluid into the eye to replace fluid aspirated from the eye in removing the lens material. Once the harder nuclear material has been removed with the assistance of ultrasonic energy the softer cortical material can be aspirated with an irrigation/aspiration cannula.

In both phases of the procedure it is important that the anterior chamber is maintained at a positive pressure and constant volume to prevent collapse thereof. Collapse of the anterior chamber can result in trauma to sensitive ocular tissues. Contact with the endothelial cells lining the posterior surface of the cornea or the iris can result in irreparable damage. Even more common is inadvertent contact or aspiration of the posterior capsule, which can result in rupture of this fine membrane. The posterior capsule prevents the escape of the fluid contained in the posterior chamber of the eye known as the vitreous humour. Rupture of the posterior capsule and loss of the vitreous increases the risk of retinal detachment and cystoid macular oedema after cataract surgery with subsequent loss of vision. Furthermore if the posterior capsule is disrupted during surgery it may not be feasible to place an intraocular lens in the preferred position in the capsular bag remnant of the original lens. This too can have a less favourable outcome than is anticipated in uncomplicated surgery. It is clearly evident, therefore, how important the maintenance of a stable pressure and volume in the anterior chamber is deemed when performing phacoemulsification.

A typical apparatus used in cataract surgery consists of a console containing a pump system used to generate vacuum and flow as well as the electrical circuitry that provides energy and control for the phacoemulsification handpiece. A conventional phacoemulsification needle delivers ultrasonic energy by a hollow bore needle which is attached to a piezoelectric crystal or magnetostrictive transducer within the handpiece which generates the ultrasonic vibrations. Transducers that produce sonic frequencies have also been utililized to reduce heat generated by ultrasonic vibrations but this mechanism is not as effective as ultrasonic energy.

The lumen of the phacoemulsification needle is attached to the aspiration line via the handpiece so that nuclear material can be aspirated during removal of the cataractous lens. The needle is surrounded by an outer sleeve that is usually manufactured from a flexible or rigid plastic material. The lumen of the sleeve is connected to the infusion line so mat fluid continuously flows around the phaco needle to replace fluid aspirated from the eye through the lumen of the needle during the procedure. The ingress of fluid through the sleeve also serves to cool the needle and prevent thermal damage induced by the vibrating needle during the application of ultrasonic energy. A phacoemulsification needle invented by the present inventor (International Patent Application WO 96/07377) describes longitudinal grooves in the wall of the needle to allow continual infusion of fluid around the needle even when the outer plastic sleeve is tightly compressed by a small sealed incision in the outer wall of the eye.

The pump systems are connected to the phacoemulsification handpiece and irrigation and aspiration cannula by tubing so that fluid and lens material can be aspirated from the eye.

There are two basic types of pump systems that achieve aspiration of fluid and lens material during phacoemulsification and cortical aspiration. The first are positive fluid displacement pumps such as a peristaltic pump. In this system fluid flow is generated in tubing and significant vacuum is achieved when the tubing is occluded. In the other system typified by a venturi pump vacuum is generated in a cassette and the subsequent flow and aspiration of fluid from the eye is related to that vacuum.

In both systems the sequence of removal of nuclear and cortical material is similar. Fluid flow is generated in the aspiration tubing and fluid is aspirated from the anterior chamber via the phacoemulsification needle or irrigation/aspiration cannula. This attracts nuclear or cortical material to the needle or cannula and occlusion of the tip or aspiration port occurs. There is then a build up of vacuum in the tubing until the negative pressure generated and the break up of the lenticular material by the application of ultrasonic energy overcomes the resistance of the lenticular material, which is then aspirated down the tubing.

Optimal fluid dynamics implies maintaining a stable pressure and volume in the anterior chamber when performing phacoemulsification. Aspiration of fluid from the anterior chamber must be balanced by adequate infusion and the desired state of fluid balance can therefore be summarized in one equation—Fᵢ=Fₒ−Inflow (Fᵢ) should equal Outflow (Fₒ). To avoid chamber collapse the pressure in the anterior chamber (P_{ac}) must also be greater than the atmospheric pressure (Pₐ) and greater than the vitreous pressure (Pᵥ)=P_{ac}>Pₐ>Pᵥ. The pressure in the anterior chamber depends on the infusion pressure which is the difference between the irrigation pressure head (Pᵢ), related to the irrigation bottle height, and the drop in pressure due to resistance to the inflow of irrigation fluid (P_{d})−Pₐ=Pᵢ−P_{d}). The anterior chamber pressure should be maintained at a constant level to avoid alterations in chamber volume which manifest as an unstable chamber during surgery. It can be seen that variables that can be manipulated to improve chamber stability are the bottle height and the cross sectional area available for the infusion of fluid. Increasing the bottle height improves the irrigation pressure head but can only partially compensate for restriction to infusion which occurs at the incision site. Ensuring that excessive outflow or aspiration from the eye is replaced by adequate infusion is therefore vital in maintaining stable pressure and volume within the anterior chamber.

Previous inventions by the present inventor which assist a surgeon in achieving this stability include novel phacoemulsification needles (International Patent Application WO 96/07377) and irrigation cannulas (International Patent Application WO 98/07398) to increase the infusional inflow as well as flow adaptive tubing (International Patent Application WO 2003/103746) to regulate the aspiration to ensure there is a balance between aspiration and infusion during a phacoemulsification procedure.

The coaxial system consisting of a central hollow phacoemulsification needle to deliver ultrasonic energy and aspirate nuclear material with a surrounding plastic sleeve to deliver infusion is very effective. A limitation of a coaxial aspiration and infusion system, however, is the required incision size. The difference in cross sectional diameter between the inner lumen of the outer sleeve and external diameter of the phacoemulsification needle determines the infusion flow rate available to replace fluid aspirated from the eye and maintain a stable anterior chamber which is critical to the safety of the procedure as described above.

The Cross sectional area of the inner diameter of an infusion sleeve can be calculated by the formula A₁=πr₁² where A₁ is the cross sectional area and r₁ is the radius of the diameter.

Similarly the Cross sectional area of the outer diameter of the phacoemulsification needle can be calculated by the formula A₂=πr₂² where A₂ is the cross sectional area and r₂ is the radius of the outer diameter of the phacoemulsification needle.

The cross sectional area available for the infusion of fluid is the difference between A₁ and A₂.

The required incision size is half the circumference of the diameter of the outer sleeve.

Incision size=1/2*C

C=Circumference of outer diameter of sleeve

C=2πr₃

R₃=radius of the outer diameter of an infusion sleeve

The flow rate of fluid within tubing is can be described by the Hagen-Poiseuille equation—Q=(P)×(π×D⁴)/(8×l×v) where Q is the volume flow rate, P is the pressure differential, D is the cross sectional diameter of the tubing, l is the length of the restricting diameter, v is the viscosity of the fluid. It can be seen that the infusion rate is proportional to the fourth power of the diameter of the infusion sleeve or the square of the cross sectional area available for infusion of fluid Thus an attempt to reduce the required incision size of a co-axial phacoemulsification needle and sleeve is limited by the reduced infusional capacity. The following table compares the cross sectional available for infusion as well as the required incision size for a range of phacoemulsification needles with diameters ranging from 0.9 mm to 1.2 mm and sleeve inner diameters ranging from 1.4 mm to 2.00 mm.

| Inner Diameter of | Outer Diameter of | Difference in cross | Required |
|---|---|---|---|
| infusion sleeve | Phaco needle | sectional area | Incision |
| (mm) | (mm) | (mm) | (mm) |
| 1.8 | 1.2 | 1.41 | 2.8 |
| 1.8 | 1.1 | 1.60 | 2.8 |
| 1.8 | 1 | 1.76 | 2.8 |
| 1.8 | 0.9 | 1.91 | 2.8 |
| 1.6 | 1.2 | 0.88 | 2.5 |
| 1.6 | 1.1 | 1.06 | 2.5 |
| 1.6 | 1 | 1.23 | 2.5 |
| 1.6 | 0.9 | 1.38 | 2.5 |
| 1.4 | 1.2 | 0.41 | 2.2 |
| 1.4 | 1.1 | 0.59 | 2.2 |
| 1.4 | 1 | 0.75 | 2.2 |
| 1.4 | 0.9 | 0.90 | 2.2 |

If an acceptable infusion rate is to be maintained a minimum incision size of 2.2 mm is achievable with a coaxial system by reducing the diameter and increasing the rigidity of the outer infusion sleeve.

Recently modifications to the control of ultrasonic energy have been introduced Typically, the ultrasonic power is varied by increasing the linear stroke length of the phacoemulsification needle by varying the ultrasonic power in a linear fashion. An alternative to the continuous control of ultrasound is to deliver energy in pulses with a duration of milliseconds or even short microseconds bursts of energy. The frequency and amplitude of the bursts can be varied by the user in a preset fashion on the console or via the footpedal control. The duty cycle or on/off times of the bursts can be fixed or variable. The interrupted nature of the application of ultrasound allows the needle to cool down during the off cycle and reduce the build up of heat.

Modulating ultrasound energy in this fashion has allowed surgeons to use a phaco needle without a sleeve and split the infusion line from the phacoemulsification handpiece to deliver the infusion via a separate cannula through a separate incision. The incision size required for a sleeveless phacoemulsification needle is less than for a coaxial system with an outer sleeve. This technique is referred to as bimanual phacoemulsification and can be accomplished with two separate 1.00 to 2.00 mm incisions. New foldable implants with a segmented thinner optic or implants manufactured from expansile elastogel materials, as described in International Patent Application WO 01/89423, have been developed and are capable of being inserted through incisions less than 2.00 mm thus taking advantage of the reduced incision size achievable with bimanual phacoemulsification.

Leakage from an incision reduces stability of the chamber during phacoemulsification. With bimanual phacoemulsification it is more difficult to prevent leakage around the bare phacoemulsification needle and in fact a small amount of leakage is helpful to cool the needle.

Conventional phacoemulsification relies on irrigation of fluid into the eye in a coaxial fashion via a conduit provided by a surrounding elastic coaxial sleeve. The surrounding sleeve also serves to reduce wound leakage by conforming to the incision in the sclera or cornea which forms the wall of the eye. In bimanual phacoemulsification the fluid is infused into the eye via a separate incision using an infusion cannula or manipulator which is typically in the form of a hollow metal tube. Similarly the phacoemulsification energy is delivered via the phacoemulsification needle. In both instances it is desirable to avoid excessive wound leakage which can compromise stability of the anterior chamber. This is accomplished by creating a very tight incision in the cornea or sclera to closely approximate the infusion cannula or phacoemulsification needle which typically have a round or less commonly an oval cross section. This results in stretching of the surrounding scleral or corneal tissue. The distortion of the corneal or scleral tissue results in poor approximation of the walls of the incision when the rigid infusion cannula or phacoemulsification needle is removed from the eye. The poor sealing may allow fluid from the exterior to gain access to the interior of the eye and increase the risk of infection or endophthalmitis following cataract surgery which may result in loss of sight.

Despite the use of pulse or burst modulation of ultrasonic energy and the use of higher vacuums to reduce the energy required to emulsify the nucleus during phacoemulsification, thermal build up and injury to the sclera and cornea can still occur particularly when removing harder cataracts.

Energy sources other than ultrasound have been considered as an alternative to ultrasound as a method to removing a cataractous lens. These include mechanical, thermal and laser methods that can also be applied in a bimanual fashion similar to the bimanual method described above with a sleeveless phacoemulsification needle. Alternative energy sources however have been found to be less efficient than ultrasound in the removal of cataracts and are not widely used.

The present invention attempts to overcome at least in part some of the aforementioned disadvantages.

During bimanual phacoemulsification, the fluid delivered into the eye to replace the aspirated fluid is delivered via a second incision. Typically the fluid is delivered via a hollow lumen cannula attached to the irrigating tubing which is connected to a bottle containing the irrigating fluid. The bottle is raised above the eye or may have air infused at a positive pressure so that the pressure in the bottle is greater than the anterior chamber. The end of the irrigating cannula may be formed into a variety of shapes to assist with manipulating and fracturing nuclear material during phacoemulsification. The terminal end of the cannula nay be open or closed with one or more side openings to allow fluid flow in to the anterior chamber of the eye. The flow rate depends on the cross sectional area of the cannula and the pressure in the irrigation line. The latter can be elevated by raising the bottle height to increase the flow rate of fluid but the critical factor which limits the infusion is the internal diameter of the irrigating cannula.

A cannula with a large diameter assists infusion but requires a larger incision. Irrigating cannulas currently in use have an internal diameter of 0.7 mm to 1.0 mm. Irrigating manipulating cannulas are more cumbersome to use than non irrigating manipulators and result in greater wound leakage. The cross sectional area of an irrigating cannula, available for infusion is significantly less than the corresponding area for infusion with a conventional phacoemulsification needle and sleeve. This factor together with the greater leakage around the wound results in reduced chamber stability which can compromise the safety of the procedure. Raising the pressure in the irrigating lens by elevating the bottle height or increasing positive air pressure in the bottle is helpful in increasing the infusion flow rate. This is achieved, however, at the expense of greater chamber pressures and the volume: of fluid used per procedure is also increased.

### SUMMARY OF THE INVENTION

In accordance with a first aspect of the present invention there is provided a phacoemulsification needle characterised by a rod member arranged for transmission of ultrasonic energy to an ocular substrate to effect emulsification thereof, and a hollow tube member having an inner surface defining a lumen arranged for aspiration of emulsified ocular material, the hollow tube member being disposed about the rod member, the rod member having a distal end and a proximal end and the hollow tube member having a distal end and a proximal end.

### DESCRIPTION OF THE DRAWINGS

The present invention will now be described, by way of example, with reference to the accompanying drawings, in which:

FIG. 1 is a perspective view of a conventional phacoemulsification needle; and

FIG. 2 is a perspective view of a solid core phacoemulsification needle in accordance with a preferred embodiment of the present invention; and

FIGS. 3 to 9 are views of phacoemulsification needles in accordance with alternative embodiments of the present invention.; and

FIG. 10 is a view of an irrigating cannula/chopper

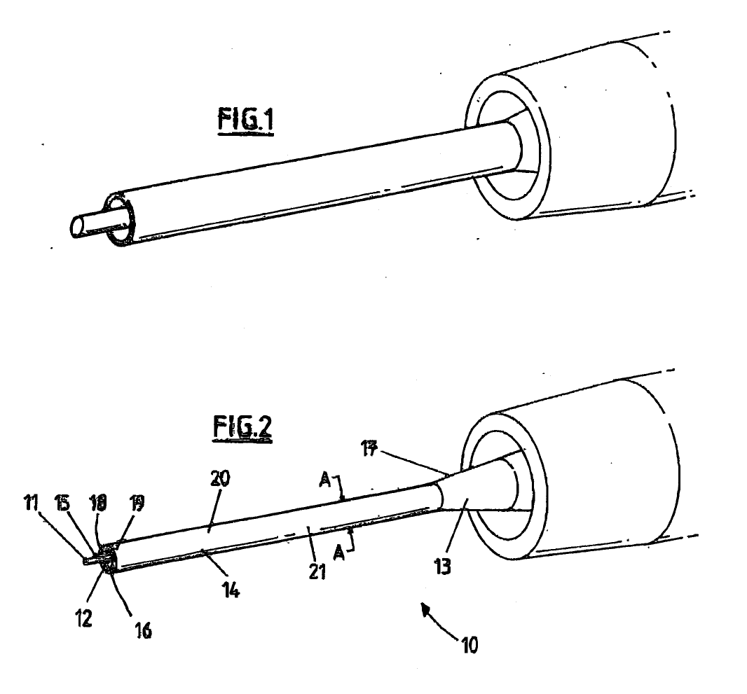

### DETAILED DESCRIPTION OF AN EMBODIMENT OF THE INVENTION

Referring to the Figures, wherein like numerals and symbols refer to like parts throughout, there is shown in FIG. 2 a solid core phacoemulsification needle 10 including an elongated rod member 12 surrounded by a hollow tube member 14 having a central longitudinal axis coincident with the rod member 12. The hollow tube member 14 comprises an elongate annular wall 16 having an inner surface 18 defining a lumen, and an outer surface 20. The hollow tube member 14 and the rod member 12 are arranged to be attached to a phacoemulsification handpiece, preferably via a threaded coupling means so that the lumen is connected to the aspiration line of the phacoemulsification console.

The rod member 12 is elongate and has a distal end 11 and a proximal end 13. In use, the rod member 12 of the phacoemulsification needle 10 is arranged to transmit ultrasonic energy to the lens material of an ocular substrate to facilitate fragmentation and emulsification of the lens material when the distal end 11 of the rod member 12 is applied thereto.

The rod member 12 is of rigid construction, generally manufactured from titanium metal.

The cross section of the rod member 12 is generally uniform throughout its length and ranges from 0.2 to 0.8 mm. Typically, the rod member 12 may have a diameter of 0.3 mm to 0.5 mm. However, the rod member 12 may be tapered such that the cross section of the proximal end 13 is greater than the cross section of the distal end 11, particularly where a threaded coupling means is located for attachment of the phacoemulsification needle 10 to the phacoemulsification handpiece.

When the rod member 12 is tapered, a decrease in the diameter of the rod member 12 may progressively occur from the proximal end 13 to the distal end 11 of the rod member 12. Alternatively, the transition to a smaller diameter may occur in one or more step like transitions along the length of the rod member 12.

The cross section of the distal end 11 of the rod member 12 may be the same, smaller or larger than the cross section of a mid-section of the rod member 12.

The cross section of the rod member 12 may be oval, circular or have a polygonal profile.

The distal end 11 of the rod member 12 may have a flat profile or may be concave, convex or hemispherical.

An elongate surface 15 disposed along the length of the rod member 12 may be smooth, ridged or grooved. A plurality of ridges or grooves on the elongate surface may be disposed in a linear or spiral pattern along the length of the rod member 12. The elongate surface 15 of the rod member 12 may also have protuberances to ensure that centration is maintained within the hollow tube member 14. The purpose of the ridges, grooves, or protruberances is to generate turbulence in the lumen of the hollow tube member 14 during aspiration of emulsified lens material. Protuberances may also act as additional surfaces to radiate ultrasonic energy. This will assist in additional emulsification and fragmentation of aspirated nuclear material as it passes down the hollow tube member and prevents possible blockages.

The hollow tube member 14 surrounds the rod member 12. The hollow tube member 14 is arranged to aspirate fluid and lens material from the eye when the rod member 12 is applied to the lens material. The aspirated fluid serves to cool and prevent thermal build up induced by the ultrasonic vibrations of the rod member 12. Infusion of fluid to the eye is delivered via a separate irrigating cannula or manipulator through a second incision. Alternatively, a non irrigating manipulator or chopper can be used or the infusion can be delivered via an anterior chamber maintainter through a third incision.

Typically, the hollow tube member 14 is manufactured from a metal material, preferably titanium, or a plastics material with an outer diameter A-A ranging from 0.9 mm to 1.2 mm, the magnitude of die outer diameter A-A being comparable with the diameter of a conventional phacoemulsification needle.

A cross section of the hollow tube member 14 extending from the outer surface 20 of the elongate annular wall 16 may be circular in cross section or oval, as an oval cross section may reduce leakage when the phacoemulsification needle 10 is inserted into a corneal or scleral incision. Alternatively distal and proximal ends 19, 17 of the hollow tube member 14 may have a circular cross section whilst a mid section 21 of the hollow tube member 14 has an oval cross section

Typically, the thickness of the annular wall 16 ranges from 0.01 mm to 0.2 mm, more generally 0.05 mm. The thickness of the annular wall 16 may be uniform throughout the length of the hollow tube member 14 or the annular wall 16 may be thinner in the mid-section 21 of the hollow tube member 14. A thinner wall in the mid-section 21 of the hollow tube member 14 may be advantageous to allow the mid-section 21 of the hollow tube member 14 to better conform to the incision so as to reduce leakage from the wound, whereas a thicker annular wall 16 in proximal and distal portions of the hollow tube member 14 would afford structural rigidity to the hollow tube member 14.

Alternatively, the thickness of the annular wall 16 disposed in the mid-section 21 of the hollow tube member 14 may be thin for a plurality of equiangularly spaced portions of the annular wall 16 with thicker portions of the annular wall 16 disposed intermediate the thinner portions to improve structural rigidity in the mid-section 21 of the hollow tube member 14. This would allow the hollow tube member 14 to deform to the shape of the incision whilst still retaining axial rigidity and preventing unwanted flexure.

Further, the hollow tube member 14 could be manufactured from both metal and plastics components. Proximal and distal portions of the hollow tube member 14 could be manufactured from relatively rigid metallic material such as titanium whilst the mid-section of the hollow tube member 14 could be composed of a flexible plastics material. Once again this mode of manufacture would allow the hollow tube member 14 to better conform to the incision, thereby reducing wound leakage. One or more longitudinal metal struts could provide continuity between the proximal and distal metallic portions of the hollow tube member 14, thereby improving rigidity and preventing unwanted flexure whilst allowing the more elastic plastic portion of the hollow tube member 14 to conform to the contours of the wound and reduce wound leakage.

An inner diameter of the hollow tube member 14 may be uniform throughout the length of the hollow tube member 14 or decreased in a tapered or step like fashion from the proximal end 17 of the hollow tube member 14 to the distal end 19 of the hollow tube member 14.

The inner surface 18 of the hollow tube member 14 is smooth or provided with a plurality of ridges, grooves, or protruberances. The ridges, grooves, or protruberances are disposed in a linear or spiral pattern along the length of the inner surface 18. The purpose of the ridges, grooves, or protruberances is to generate turbulence in the lumen of the hollow tube member 14 during aspiration of emulsified lens material.

The outer surface 16 of the hollow tube member 14 can be rubberized to help produce a seal and reduce wound leakage. Similarly a flexible sleeve may be applied to the outer surface 16 to help reduce wound leakage. Although it is envisaged that the phacoemulsification needle 10 would have maximum utility when used with a separate incision for irrigation, the phacoemulsification needle 10 could be used with an outer sleeve for irrigation in a co-axial irrigation aspiration system.

The distal end 19 of the hollow tube member 14 may be provided with a rounded or a flat edge that is smooth or sharp. The edge of the distal end 19 of the hollow tube member 14 may be thinner, thicker, or the same thickness as the annular wall 16 of the hollow tube member 14. The edge is typically bevelled or contoured to a concave surface to improve a sealing effect when suction is applied to a fragment of lens material.

The distal end 11 of the rod member 12 may project outwardly from the distal end 19 of the hollow tube member 14, be substantially laterally aligned with the distal end 19 of the hollow tube member 14, or be disposed inwardly from the distal end 19 of the hollow tube member 14.

The rod member 12 and the hollow tube member 14 are typically constructed separately to minimize transmission of vibration from the rod member 12 to the hollow tube member 14. Nevertheless it is envisaged that the phacoemulsification needle 10 could be manufactured as a single piece construction.

An advantage of the present invention is that it can be used with a conventional handpiece with modified connections. Conventionally, the aspiration line of a phacoemulsification handpiece is in fluid communication with the lumen of a prior art phacoemulsification needle. In relation to the phacoemulsification needle 10 of the present invention, the aspiration line is occluded by the rod member 12 that obstructs the usual aspiration conduit in a conventional phacoemulsification handpiece. Instead the aspiration line is disposed in fluid communication with the previous infusion line of the phacoemulsification handpiece so that aspiration occurs around the rod member 12 and is laterally confined by the lumen defined by the inner surface 18 of the hollow tube member 14. Typically, the aspiration line is connected by a female connector on the terminal end of the aspiration tubing to a corresponding male connector on the phacoemulsification handpiece. It is necessary to replace the female connector on the aspiration tubing with a male connector so that it can be attached to the female connector on what used to be the irrigation channel of a conventional phacoemulsification handpiece. Alternatively, an intermediate connector can be used to convert the female connection at the terminal end of the aspiration tubing into a male connector or the female connector of the previously used irrigating channel into a male termination.

As described previously the irrigating tubing is attached to a separate cannula or irrigating chopper that is inserted into the eye via a separate incision.

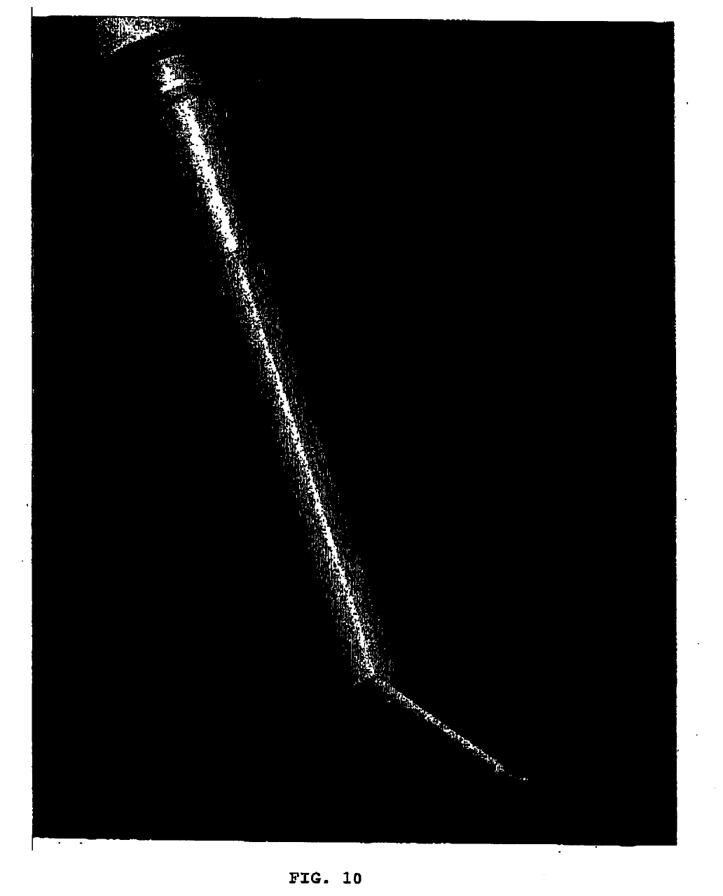

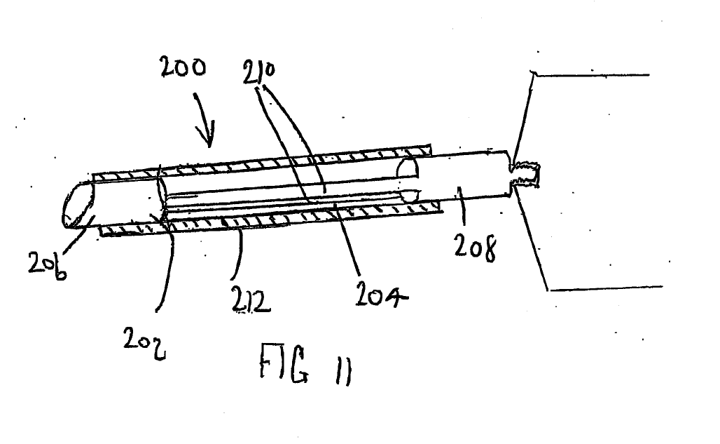

The same type of constructions described above with respect to the hollow tube member 14 can be used in manufacturing an irrigation cannula or irrigating chopper which provides the required infusion for the ultrasonic rod member 12 when used in bimanual phacoemulsification. Conventional irrigating cannulas/choppers are manufactured from metal to provide maximum infusion as well as structural rigidity which is important in manipulating and fracturing fragments of nucleus in conjunction with the ultrasonic probe. A typical irrigating cannula/chopper is illustrated in FIG. 10.

A metal cannula however can result in excessive leakage of fluid. Providing an irrigation cannula with a more flexible mid portion to reduce wound leakage whilst retaining adequate axial strength would be advantageous and can be manufactured in the same manner as described for the outer tube for the ultrasonic probe as described above.

Similarly, the cross sectional profile of an irrigating cannula may be circular or oval in cross section. An oval cross section is helpful in reducing wound leakage. Alternatively, the distal and proximal ends of the cannula may have a circular cross section whilst the mid section has an oval cross section.

The present invention provides a phacoemulsification needle 10 that has an inbuilt cooling mechanism that does not require an infusion sleeve. The phacoemulsification needle 10 is designed to be used with a separate incision for infusion via an infusion cannula, irrigating chopper or an anterior chamber maintainer. The phacoemulsification needle 10 is therefore effective for bimanual phacoemulsification procedures as it can be used via a microincison in the range of 0.8 to 2.00 mm without risk of thermal damage to the sclera or corneal incision.

The phacoemulsification needle 10 provides more efficient application of energy than a conventional needle, improved cooling with less chance of thermal injury and safer application of ultrasound with a sealed wound.

As described the hollow tube member 14 which surrounds the rod member 12 may be manufactured from a plastics or an elastic material to better conform to the incision in the wall of the eye which provides a better seal with reduced wound leakage during surgery. Furthermore the reduced stretching of the surrounding tissue will result in better approximation of the incision after completion of the procedure. The hollow tube member 14 does not provide infusion as with a conventional coaxial phacoemulsification needle but forms a conduit for the aspiration of fluid.

There is also provided in the present invention a manipulator having a distal end for manipulating and fracturing nuclear material, and a proximal end attached to an irrigating handle, preferably via a threaded coupling means. The distal end may be shaped in several different ways to assist manipulating and fracturing nuclear material.

The manipulator is of solid construction, preferably fabricated from a metal such as titanium and has a small cross-sectional diameter in the range of 0.3 to 0.5 mm. A separate sleeve is attached to the distal end of the irrigating handle and co-axially surrounds the manipulator.

An irrigation line is attached to the proximal end of the handle and fluid is delivered into the eye via the sleeve in a coaxial fashion. The advantage of this system compared to an irrigating cannula is that the sleeve can conform to the wound and prevent excessive wound leakage. Furthermore, a flexible sleeve with an internal diameter in the range of 1.1 to 1.4 mm can deliver as much fluid into the eye as a conventional co-axial phacoemulsification needle and sleeve. The incision size of the sleeved irrigating manipulator however is significantly less than a coaxial phacoemulsification needle and sleeve. Although the diameter of a sleeved irrigating manipulator used with bimanual phacoemulsification is slightly larger than a cannula style irrigating manipulator it is less cumbersome man an irrigating cannula of an equivalent diameter required to deliver the same infusion flow rate. An irrigating sleeved manipulator therefore has improved infusion and ergonomics as well as reduced wound leakage compared to an irrigating cannula style manipulator when used in conjunction with the solid core phacoemulsification needle of the present invention in bimanual phacoemulsification procedures. A terminal end of the cannula may be modified to form a protrusion which is adapted in use to fracture or manipulate fragments of nucleus.

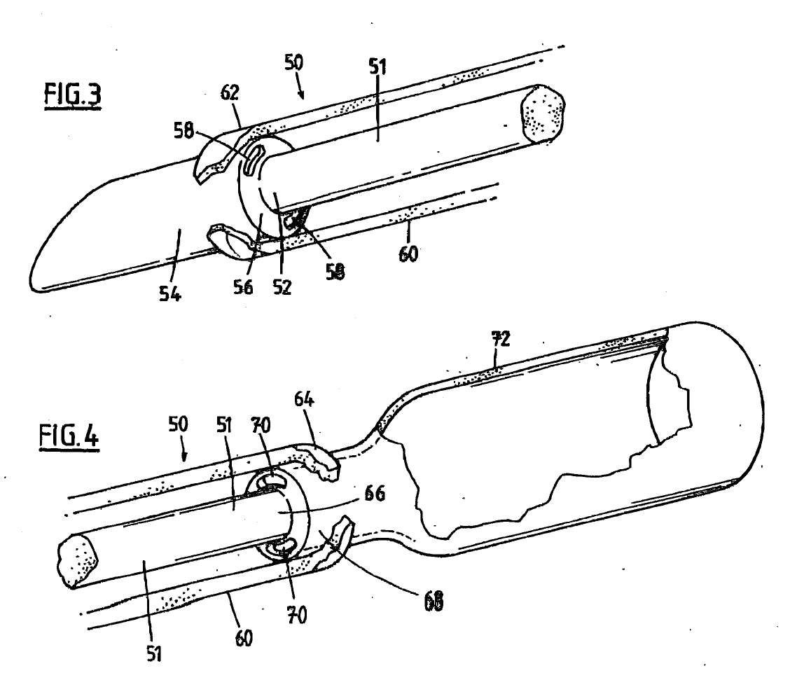

In FIGS. 3 and 4 of the accompanying drawings there is shown a phacoemulsification needle 50 which comprises a solid shaft 51 having a distal end 52. A cup-shaped member 54 having a base 56 is abutted to the distal end 52. As shown, the base 56 is formed with a pair of apertures 58 outwardly of the shaft 51.

A hollow tubular member 60 is mounted about the shaft 51 and extends from a distal end 62 adjacent the distal end 52 of the shaft 51 to a proximal end 64 located adjacent a proximal end 66 of the shaft 51. The proximal end 64 is mounted about an enlarged proximal extension 68 of the shaft 51. The proximal extension 68 extends outwardly from the main portion of the shaft 51 and has a pair of apertures 70 outwardly of the shaft 51. The member 60 defines a lumen with the shaft 51.

Further, the proximal extension 68 broadens out into a wide portion 72 which is arranged to be coupled with a phacoemulsification hand piece (not shown).

In use, fragmented or emulsified ocular material, such as lens material, is aspirated through the cup-shaped member 54 and the apertures 58 into the lumen defined by the hollow tubular member 60 and men through the apertures 70 into the proximal extension 68 and the wide portion 72.

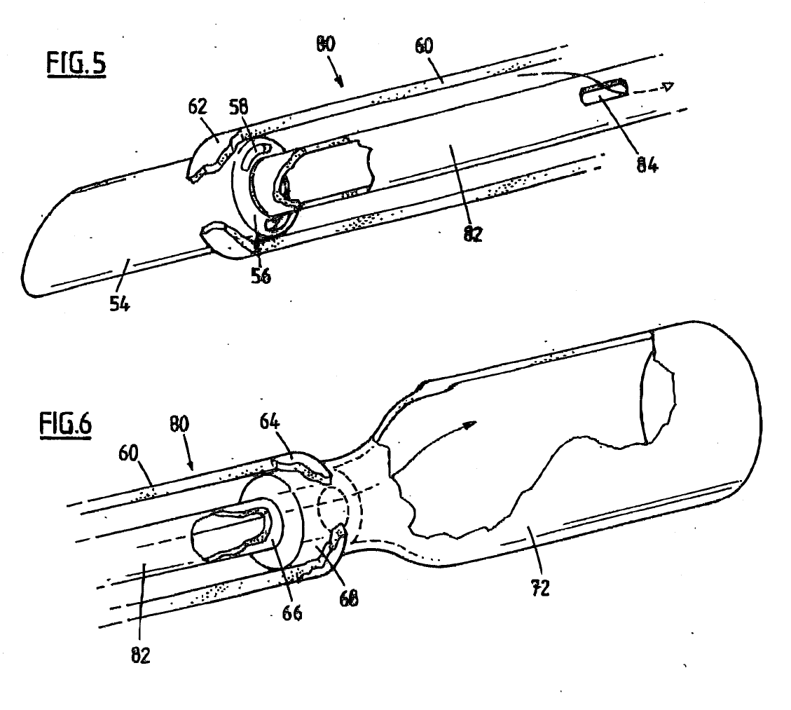

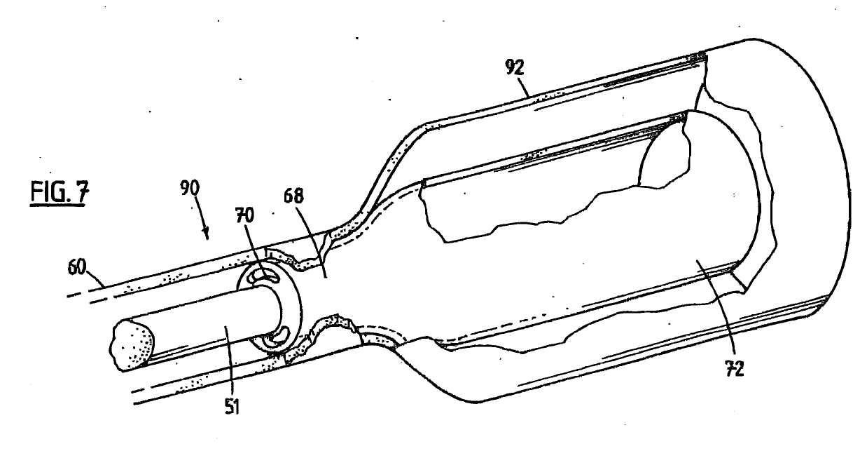

In FIGS. 5 and 6 of the accompanying drawings, there is shown a phacoemulsification needle 80 which is similar to the needle 50 and like reference numbers refer to like parts. There is also provided a shaft 82 which is hollow. In this case ocular material is aspirated through the cup shaped member 54 into the shaft 82 and then into the proximal extension 68 and the wide portion 72. Additional ocular material is aspirated through the apertures 58 into the lumen of the hollow tube member 60.

Further, the shaft 82 is provided with a pair of cutaways 84 (only one of which can be seen in FIG. 5). Ocular material in the lumen is aspirated into the shaft 82 through the cutaways 84 and then into the proximal extension 68.

In FIG. 7 of the accompanying drawings there is shown a phacoemulsification needle 90 which is similar to the needle 50 and like reference numbers refer to like parts. The shaft 51 is, in this case, solid as shown in FIGS. 3 and 4. Also, the distal end of the needle 90 is as shown in FIG. 3.

In this case however, the hollow tube member 60 extends proximally towards a wide proximal portion 92 adjacent the proximal extension 68 of the needle 90. The proximal portion 92 is arranged to be connected to a phacoemulsification hand piece. Ocular material may be aspirated along the length of the tubular member 60 as far as the apertures 70. Further, ocular material is initially aspirated through the cup-shaped member 54 and then through the apertures 58 into the lumen defined by the tubular member 60. The ocular material from the apertures 70 enters the wide portion 72.

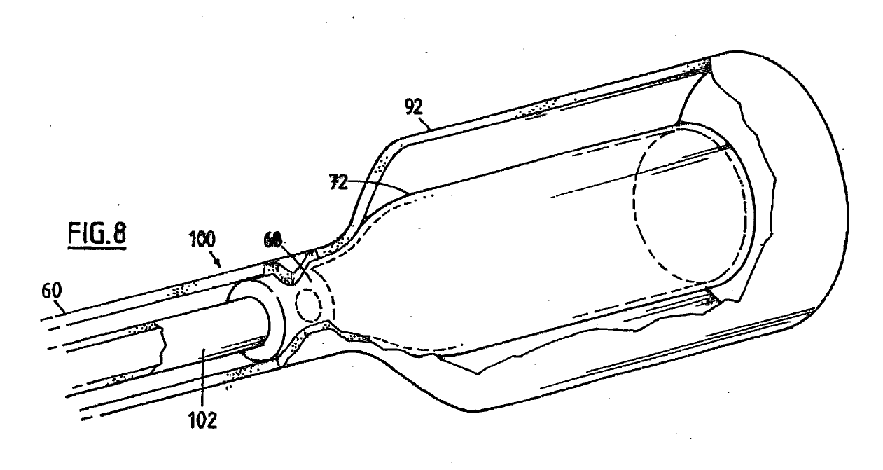

In FIG. 8 of the accompanying drawings there is shown a phacoemulsification needle 100 which is similar to the needle 90 and like reference numerals denote like parts. In this case there is provided a shaft 102 which is hollow. Thus, aspirated ocular material can be aspirated through the cup-shaped member 54 along the entire length of the shaft 102 and into the proximal extension 68 and also the lumen through the apertures 58. Also, cutaways similar to the cutaway 84 shown in FIG. 5 in the shaft 102 enable ocular material to be drawn from the lumen into the shaft 102.

It is envisaged that in the embodiment of FIG. 8 a plug could be inserted in the wide portion 72 to block off the shaft 102. In this case, ocular material would be aspirated from the lumen or the shaft 102 into the wide proximal portion 92.

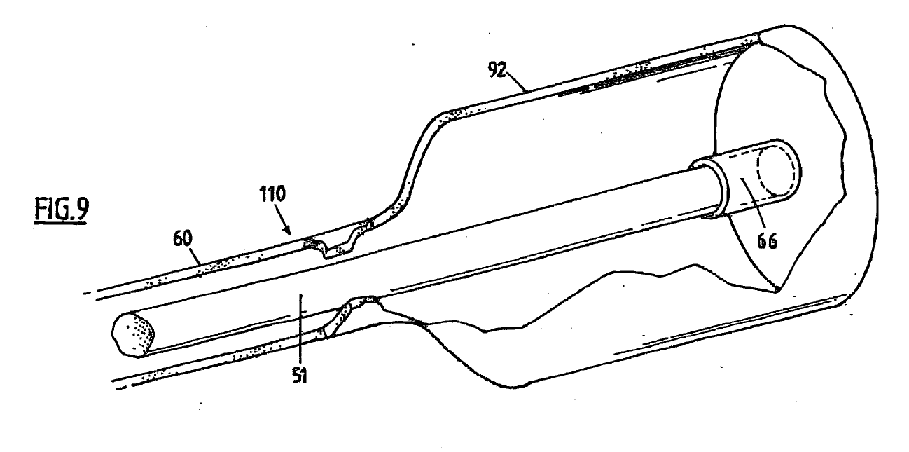

In FIG. 9 there is shown a phacoemulsification needle 110 which is similar to the needle 50 except that the shaft is solid throughout and there is a widened proximal portion 92. Ocular material is aspirated directly from the lumen into the widened proximal portion 92.

The handpiece contains the piezoelectric element for generating ultrasonic energy and is attached to the aspiration line connected to the phacoemulsification console.

A similar construction can he employed for a cannula to deliver infusion into the eye at a separate incision. Here the hollow tube member 14 and the rod member 12 create a single channel for infusion of fluid. The cannula may be attached to or be continuous with a handle which is in turn attached to the irrigating line.

Modifications and variations such as would be apparent to a skilled addressee are deemed within the scope of the present invention.

For example, it is possible to create a phacoemulsification needle 200 in accordance with the presence invention in which a hollow phacoemulsification needle 202 has a central portion 204 in which a substantial proportion of the wall of the needle is removed. Thus, the distal end 206 of the needle 202 is connected to the proximal end 208 by two struts 210.

Further, a hollow tubular member 212 is placed around the needle 202 to form a lumen in conjunction with the needle 202.

As shown the hollow tube 212 can terminate at the proximal end 208 of the needle. Alternatively it can extend rearwardly and be attached to the phacoemulsification handpiece.

Thus, in this embodiment the entire phacoemulsification needle is constituted by the hollow needle 202 with the cutaway portions and the hollow tubular member 212. This arrangement has the advantage that when inserted into a wound the central portion of the needle can readily be compressed to conform closely to the shape of the incision.

In other respects the phacoemulsification needle of FIG. 11 operates in similar manner to the other embodiments of the present invention described hereinabove.

1. A phacoemulsification needle characterised by a rod member arranged for transmission of ultrasonic energy to an ocular substrate to effect emulsification thereof, and a hollow tube member having an inner surface defining a lumen arranged for aspiration of emulsified ocular material, the hollow tube member being disposed about the rod member, the rod member having a distal end and a proximal end and the hollow tube member having a distal end and a proximal end.

2. A phacoemulsification needle according to claim 1, characterised in that the rod member is concentric with a central longitudinal axis of the lumen,

3. A phacoemulsification needle according to claim 1 or 2, characterised in that the rod member is of uniform cross section throughout its length.

4. A phacoemulsification needle according to claim 1 or 2, characterised in that the rod member is tapered throughout its length so that the cross section of the proximal end is greater than the cross section of the distal end.

5. A phacoemulsification needle according to any one of the preceding claims, characterised in that the rod member is of circular, oval or polygonal shape in cross section.

6. A phacoemulsification needle according to any one of the preceding claims, characterised in that the rod member has a distal end which is flat, concave, convex or hemispherical.

7. A phacoemulsification needle according to any one of the preceding claims, characterised in that the rod member has an elongate outer surface which is ridged, grooved or provided with protuberances to generate turbulence in the lumen in use.

8. A phacoemulsification needle according to any one of the preceding claims, characterised in that hollow the tube member is circular or oval in cross section.

9. A phacoemulsification needle according to any one of claims 1 to 7, characterised in that the distal and proximal ends of the hollow tube member have a circular cross section whilst a mid section thereof has an oval cross section.

10. A phacoemulsification needle according to any one of the preceding claims, characterised in that the hollow tube member has an outer surface and the inner surface which define an annular wall.

11. A phacoemulsification needle according to claim 10, characterised in that the annular wall is of uniform thickness throughout.

12. A phacoemulsification needle according to any one of claim 10, characterised in that the annular wall has a mid section which is thinner than the thickness of the distal and proximal ends thereof.

13. A phacoemulsification needle according to claim 12, characterised in that the mid section is thin in a plurality of spaced portions of the annular wall with thicker portions disposed between the thinner portions.

14. A phacoemulsification needle according to any one of the preceding claims, characterised in that the hollow tube member has distal and proximal portions formed of relatively rigid metallic material whilst a mid section is formed of relatively flexible plastics material.

15. A phacoemulsification needle according to any one of the preceding claims, characterised in that the inner surface of the hollow tube member is provided with ridges, grooves or protuberances to generate turbulence in the lumen in use.

16. A phacoemulsification needle according to any one of the preceding claims, characterised in that the hollow tube member has a rubberised exterior or a flexible sleeve applied to an exterior surface thereof to reduce wound leakage.

17. A phacoemulsification needle according to any one of the preceding claims, characterised in that the phacoemulsification needle is arranged for use in bimanual phacoemulsification procedures.

18. A phacoemulsification needle according to any one of the preceding claims characterised in that the rod member is solid throughout.

19. A phacoemulsification needle according to any one of the claims 1 to 17, characterised in that the rod member is at least partially hollow.

20. A phacoemulsification needle according to claim 19, characterised in mat the rod member has a hollow distal end to facilitation aspiration of ocular material.

21. A phacoemulsification needle according to claims 19 or 20, characterised in that the proximal end of the rod member is hollow to facilitate aspiration of ocular material.

22. A phacoemulsification needle according to claim 21, characterised in that the hollow distal and proximal portions are broader in cross section than an intermediate portion of the rod member.

23. A phacoemulsification needle according to claim 19, characterised in that the proximal end of the rod member is enlarged so as to be compatible with a phacoemulsification hand piece.

24. A phacoemulsification needle according to claim 21, characterised in that the rod member has an intermediate portion which is hollow so that the rod member is hollow throughout.

25. A phacoemulsification needle according to claim 24, characterised in that the hollow tube member is arranged to divert aspirated ocular material into the proximal end of the rod member.

26. A phacoemulsification needle according to claim 24, characterised in that aspirated ocular material is arranged to be diverted through the rod member and the hollow tube member.

27. A phacoemulsification needle according to claim 20, characterised in that the proximal end of the rod member is solid.

28. A phacoemulsification needle according to claim 20,characterised in mat the rod member is solid except at the distal end.

29. An irrigation cannula or irrigating chopper characterised by a hollow tube member extending about the cannula or chopper, which hollow tube member has a flexible mid portion to reduce wound leakage.
